SEQUENCE LISTING (2 sequences)

SEQ ID NO: 1 - protein, 9 aa, Artificial Sequence
  Description of Artificial Sequence/note = Synthetic Construct
XHWSYXLRX

SEQ ID NO: 2 - protein, 9 aa, Artificial Sequence
  VARIANT 1: Xaa = oxo Pro
XHWSYXLRX

(19) United States
(12) Patent Application Publication
(10) Pub. No.: US 2004/0248971 A1
(43) Pub. Date: Dec. 9, 2004

(54) Use of vitamin e succinate and antiandrogen combination

(76) Inventors: Shuyuan Yeh, Rochester, NY (US); Chawnshang Chang, Pittsford, NY (US)

Correspondence Address: NEEDLE & ROSENBERG, P.C., SUITE 1000, 999 PEACHTREE STREET, ATLANTA, GA, 30309-3915, US

(21) Appl. No.: 10/484,785

(22) PCT Filed: Jul. 29, 2002
(86) PCT No.: PCT/US02/23999

Related U.S. Application Data
(60) Provisional application No. 60/308,295, filed on Jul. 27, 2001.

(51) Int. Cl. A61K 31/355

(52) U.S. Cl. 514/458

(57) ABSTRACT

Disclosed are compositions and methods related to vitamin E and prostate cancer.

### I. RELATED APPLICATIONS

This application claims priority of United States Provisional Application No. 60/308,295 filed on Jul. 27, 2001, for “Vitamin E Inhibition of Androgen Receptor and the Expression of Prostate Specific Antigen in Prostate Cancer Cells” by Yeh et al. This application is incorporated in its entirety by reference herein.

### II. ACKNOWLEDGEMENTS

This work was supported by National Institutes of Health Grant DK60912. The federal government may have rights in this invention.

### III. BACKGROUND OF THE INVENTION

Prostate cancer is the most common cancer and second leading cause of cancer deaths in American men.

A notable gene regulated by androgen is prostate specific antigen (PSA). PSA has been demonstrated as a sensitive and selective marker for prostate cancer screening and assessment, therefore, PSA is used as an indicator of disease and response to prostate cancer therapy.

It is shown herein that VES decreases intracellular and secreted levels of PSA in human prostate cancer LNCaP cells, which have been cultured either under normal serum or androgen-stimulated conditions. Furthermore, these results indicated that inhibition of PSA is concomitant with VES-mediated down-regulation of AR protein levels. In addition, inhibition of the AR protein by VES arises at the protein level and not mainly at the level of transcriptional regulation level of AR mRNA.

### IV. SUMMARY OF THE INVENTION

In accordance with the purposes of the compositions and methods disclosed herein, these compositions and methods, in one aspect, relate to compositions and methods for altering PSA levels in cells or in a subject.

Additional advantages of the invention will be set forth in part in the description which follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### V. BRIEF DESCRIPTION OF THE DRAWINGS

The accompanying drawings, which are incorporated in and constitute a part of this specification, illustrate several embodiments of the invention and together with the description, serve to explain the principles of the invention.

FIG. 1 shows VES inhibits the cell growth of LNCaP cells, but not prostate fibroblast. (A) LNCaP cells were cultured in 8% CS-FBS RPMI and treated with DHT (5 nM), Suc (10 μM), HF (5 μM), VES (1 μM or 10 μM), or VES (10 μM) combined with HF (5 μM). Cells were harvested at the time indicated. (B) LNCaP cells were cultured in 8% FBS RPMI and treated with Suc (10 μM), HF (5 μM), VES (1 μM or 10 μM), or VES (10 μM) combined with HF (5 μM). Cells were harvested at the time indicated. (C) Phase-contrast photomicrographs depict representative morphological responses of LNCaP cells at 2, 4, and 6 days of exposure to ethanol, 10 μM Suc, or 10 μM VES. (×100.) (D) Primary cultured prostate fibroblast cells were maintained in 10% FBS DMEM and treated with Suc and VES as indicated. Cell growth was determined by the MTT assay. The control group was cultured in 0.1% (vol/vol) ethanol and was set at 100%. All results were compared with the control group at the same time point.

FIG. 2 shows VES inhibits the expression of PSA. (A) VES inhibits PSA expression at the protein level. LNCaP cells were cultured in 8% FBS RPMI or 8% CS-FBS RPMI plus 5 nM DHT and treated with ethanol, 10 μM Suc, or 10 μM VES (0.1% vol/vol) for 2 and 4 days. Cells without treatment were harvested on day 2 and used as a control. Western blotting was used to detect the expression of PSA protein. Actin served as an internal control. (B) VES inhibits PSA expression at the mRNA level. LNCaP cells were treated with 10 μM VES, 10 μM Suc, or ethanol (0.1% vol/vol), respectively. Cells were harvested on days 1, 2, and 3 for Northern blotting analysis. β-Actin served as an internal control. (C) VES inhibits the expression of PSA gene at the transcription level. A transient transfection assay was performed in LNCaP cells using the PSA6.0-Luc plasmid with treatment of 10 μM Suc, 10 μM VES, or ethanol (0.1% vol/vol). The histogram represents the level of luciferase activity normalized to simian virus 40 activities and expressed as the fold of the PSA-promoter activity without VES treatment in the presence of DHT. (D) VES has no effect on the transactivation activity of SP1. In COS-1 cells, 1 μg of Gal4-DBD-fused SP1 (Gal4-SP1) was cotransfected with 1 μg of pG5-Luc and 5 ng of SV40RL in the presence or absence of 10 μM VES as indicated. The transfections were performed at least three times and presented as an average±SD. FIG. 2E shows VES inhibits PSA expression in protein level. In RPMI with 8% FBS medium, LNCaP cell was treated with succinic acid (10⁻⁵M), VES (10⁻⁵M) and Vitamin D₃ (10⁻⁸) for 2, 4, and 6 days. LNCaP cells without any treatment harvested on the first day (day 0) is used as a control. Western blotting was applied to detect the expression of PSA protein level. In RPMI medium with 8% CS-FBS, LNCaP cell was treated with succinic acid (10⁻⁵M, VES (10⁻⁵M and Vitamin D₃ (10⁻⁸) for 2, 4, and 6 days. DHT (5×10⁻⁹M) was supplied daily. LNCaP cells without any treatment harvested on the first day (day 0) and LNCaP cells treated with succinic acid (10⁻⁵M) for 4 days without DHT were used as control. Western blotting was used to detect the expression of PSA protein level. (F). VES inhibits PSA expression in RNA level. LNCaP cells were cultured in RPMI medium with 8% FBS or medium with 8% CS-FBS plus DHT (5×10⁻⁹M) daily, treated by VES (10⁻⁵M), succinic acid (10⁻⁵M) and Vit D₃ (10⁻⁸M) respectively. Cells were harvested on day 1, 2, and 3 for Northern blotting analyses. (G). VES inhibits the expression of PSA gene at the transcriptional level. A transient transfection was performed in LNCaP cells using PSA(6.0)-Luc plasmid and treated with ethanol (0.1% v/v), succinic acid (10⁻⁵M), VES (10⁻⁵M) and Vit D₃ (10⁻⁸M) respectively. The transfections were performed three times and presented as an average: bar-denotes standard deviation.

FIG. 3 shows VES differentially regulates the protein level of AR, VDR, PPARα, and RXRα. (A) VES down-regulates AR at the transcription and posttranscription level. LNCaP cells were cultured in 8% FBS RPMI and treated with 10 μM VES, or ethanol (0.1% vol/vol). Cells were harvested at different time points. Twenty-five micrograms of RNA and 50 μg of protein collected from the same culture dish were applied for Northern blotting and Western blotting assays, respectively (45). The amount of actin is shown as a control. (B) LNCaP cells were treated with 10 μM Suc, 10 μM VES, or ethanol (0.1% vol/vol). LNCaP cells without treatment were harvested on day 2 and used as a control. Whole-cell lysates were subjected to Western blotting assay using primary antibodies for AR, VDR, PPAR, or RXR.

FIG. 4 shows VES cannot affect the ligand binding and N-C dimerization of AR. (A) LNCaP cells cultured in 8% CS-FBS RPMI were treated with 2.5 nM [³H]R1881, with or without 100-fold excess of unlabeled R1881. Cells were harvested and washed, and the radioactivity was measured. [³H]R1881-binding without competition was set at 100%. Data were presented as means±SD and from the values of at least three independent experiments. (B) AR N-C dimerization. COS-1 cells without endogenous AR were cotransfected with GAL4-DBD-fused AR-HLBD (Gal4-AR-HLBD), VP16-fused AR-N (VP16-AR-N), or pSG5-SRC-1 in the presence or absence of 10 nM DHT and/or 10 μM VES. HF was added as a control to block DHT-mediated AR N-C interaction. SRC-1, a steroid receptor coactivator, was applied as a positive control to enhance N-C interaction (26).

FIG. 5 shows VES has no effect on AR protein stability, but reduces AR translation. (A) For the stability assay, after pretreatment with ethanol or 10 μM VES (0.1% vol/vol) for 24 h, LNCaP cells were labeled with [³⁵S]methionine. After 2-h labeling, cells were washed and supplied with fresh medium, and then were harvested at time points of 0, 2, 6, and 12 h. (B) For the AR-translation assay, LNCaP cells were cultured in methionine-free medium for 2 h, then 100 μCi/ml of [³⁵S]methionine was added and remained in the medium until harvesting at 0.5, 2, 6, and 12 h. After cell lysis, 300 μg of total protein was subjected to immunopreciptitation by anti-AR NH27 antibody, resolved on an SDS/8% PAGE gel, and the autoradiographic signal was quantitated by using IQMAC software (Molecular Dynamics).

FIG. 6 shows that SM has no effect on AR and PSA expression. LNCaP cells were cultured in 8% FBS RPMI and treated with 10 μM SM, 10 μM VES, or ethanol (0.1% vol/vol). Protein harvested from cells without treatment on the first day (day 0) was used as a control. Fifty micrograms of whole-cell lysate was subjected to Western blotting assay.

FIG. 7A shows α-VES accelerates the degradation rate of AR. LNCaP cells were cultured on 100-mm dishes for 48 h. 2 h before [³⁵S]-methionine labeling; the cells were starved with methionine-free medium. Then, 100 μCi/ml of [35S]-methionine was added into the medium for 1 h. The cells were washed by PBS and supplied with fresh medium including 8% FBS, and then harvested at time points of 0, 2, 6, and 12 h. After cell lysis, 150 μg of total protein was subjected to immunopreciptitation by AR-NH27 antibody, resolved on 10% SDS-PAGE gel, and autoradiography. FIG. 7(B) shows α-VES slows down the accumulation of AR protein. LNCaP cells were seeded and methionine-starvated as above. 100 μCi/ml of [³⁵S]-methionine was then added into medium and remained in the medium until harvesting. The cells were harvested at 0.5, 2, 6, and 12 h. 150 μg of total cell extract was then subjected to immunoprecipitation, gel resolution, and autoradiography as above.

FIG. 8 shows the effects of α-Vit E, γ-Vit E, and VES on the growth of LNCaP cells. LNCaP cells were cultured in RPMI medium with 8% FBS, and treated with 10⁻⁵M α-Vit E, γ-Vit E, or VES. Cells were harvested at the time indicated in the figure. All the cell growth was determined by cell counting and MTT assay. Control group contained 0.1% (v/v) ethanol and was set at 100%. All results were compared with control group at the same time point.

FIG. 9 shows the effects of VEA, VES, α-Vit E, and γ-Vit E on AR and PSA expression. LNCaP cells were cultured in RPMI medium with 8% FBS, and treated with 20⁻⁵M of the indicated reagent. Cells were harvested at the time indicated in the figure. Proteins harvested from cells without treatment on day 2 were used as a control. 60 micrograms of whole cell lysate was subjected to Western blotting assay.

FIG. 10 shows the effects of α-Vit E, γ-Vit E, VES and VEA on the growth of LNCaP cells. LNCaP cells were cultured in RPMI medium with 8% FBS, and treated with 10⁻⁵M of the indicated reagent Cells were harvested at the time indicated in the figure. All the cell growth was determined by cell counting and MTT assay. Control group contained 0.1% (v/v) ethanol and was set at 100%. All results were compared with control group at the same time point.

### VI. DETAILED DESCRIPTION

The present compositions and methods disclosed herein may be understood more readily by reference to the following detailed description of preferred embodiments of the subject matter and the Examples included therein and to the Figures and their previous and following description.

Before the present compounds, compositions, articles, devices, and/or methods are disclosed and described, it is to be understood that this invention is not limited to specific synthetic methods, specific recombinant biotechnology methods unless otherwise specified, or to particular reagents unless otherwise specified, as such may, of course, vary. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Thus, for example, reference to “a pharmaceutical carrier” includes mixtures of two or more such carriers, and the like.

Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed.

In this specification and in the claims which follow, reference will be made to a number of terms which shall be defined to have the following meanings:

“Optional” or “optionally” means that the subsequently described event or circumstance may or may not occur, and that the description includes instances where said event or circumstance occurs and instances where it does not.

Abbreviation: AR, androgen receptor, PSA, prostate specific antigen; DHT, 5α-ihydrotestosterone; Su, Succinic acid; α-VES, α-Vitamin E succinic acid, HF, hydroxyflutamide; Vit D₃, 1α, 25-hydroxyvitamin D₃; VDR, vitamin D receptor; PPAR, peroxisome proliferator-activated receptor, RXR, retinoid X receptor; H-LBD, hinge and ligand binding domain; Luc, luciferase; CAT, chlorenphenical acetyltransferase, FBS, fetal bovine serum, LH-RH—Leutinizing hormone—releasing hormone, BPH—Benign prostatic hyperplasia, DES—diethylstilbesterol, and GnRH—Gonadotropic releasing hormone.

A. Compositions

Disclosed are the components to be used to prepare the disclosed compositions as well as the compositions themselves and to be used within the methods disclosed herein. These and other materials are disclosed herein, and it is understood that when combinations, subsets, interactions, groups, etc. of these materials are disclosed that while specific reference of each various individual and collective permutation of these compounds may not be explicitly disclosed, each is specifically contemplated and described herein. For example, if a particular VES or VES derivative are disclosed and discussed and a number of modifications that can be made to a number of molecules including the VES or VES derivative are discussed, specifically contemplated is each and every combination and permutation of VES or VES derivative and the modifications that are possible unless specifically indicated to the contrary. Thus, if a class of molecules A, B, and C are disclosed as well as a class of molecules D, E, and F and an example of a combination molecule, A-D is disclosed, then even if each is not individually recited each is individually and collectively contemplated meaning combinations, A-E, A-F, B-D, B-E, B-F, C-D, C-E, and C-F are considered disclosed. Likewise, any subset or combination of these is also disclosed. Thus, for example, the sub-group of A-E, B-F, and C-E would be considered disclosed. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods.

Disclosed are compositions comprising VES and VES derivatives. Also disclosed are compositions comprising VES or VES derivatives and an antiandrogen. Pharmaceutical compositions comprising VES or VES derivatives and pharmaceutical compositions comprising VES or VES derivatives and an antiandrogen are also disclosed.

Antiandrogens typically are compositions that inhibit the activity of androgen receptor and include for example hydroxyflutamide (HF). Preferred are antiandrogens that function as HF. Also preferred are antiandrogens that function as HF and which are structurally related to HF.

Disclosed herein—tocopheryl succinate (VES) can suppress the expression of prostate-specific antigen (PSA), a marker for the progression of prostate cancer. VES can also suppress androgen receptor (AR) expression by means of transcriptional and posttranscriptional modulation, but not ligand binding, nuclear translocation, or AR dimerization. This VES-mediated inhibition of AR is selective because VES does not repress the expression of other nuclear receptors. Cell growth studies further show that VES inhibits the growth of prostate cancer LNCaP cells. In contrast, hydroxyflutamide (HF), an antiandrogen currently used to treat prostate cancer patients, only slightly inhibits LNCaP cell growth. Interestingly, simultaneous addition of HF and VES results in a more significant inhibition of LNCaP cell growth. Moreover, selenomethionine (SM), a prostate cancer treatment adjuvant, shows an inhibitory effect on LNCaP cell growth, yet has no effect on the AR/PSA pathway. Together, this data indicate that VES can suppress androgen/AR-mediated cell growth and PSA expression by inhibiting AR expression at both the transcription and translation levels.

Prostate cancer is the most common noncutaneous cancer and second leading cause of cancer death in American men (8). The androgen receptor (AR) is required for the development of both the normal prostate gland and prostate cancer. AR is a critical factor in the development and differentiation of the prostate gland and prostate cancer. In the later stages of prostate cancer, more than 80% of prostate cancer tissues remain positive for AR staining (34). Overall, these observations indicate the importance of the AR in the initiation and progression ofprostate cancer. In the early stages of prostate cancer, almost all cancer cells are androgen-dependent and highly sensitive to anti-androgens. However, prostate cancer usually recurs after a few years of androgen ablative treatment, and most cancer cells become androgen-independent, rendering antiandrogen therapy useless (9). Reports suggest that mutations in the AR ligand-binding domain, AR coregulators, or receptor phosphorylation may enable the AR to respond to nonandrogen agonists (10-13). Furthermore, the activation of the AR by these factors during androgen ablation therapy may facilitate androgen-independent prostate cancer growth. As androgen-independent prostate tumors are incurable, the prevention of such aberrant AR activation is an attractive therapeutic target. Prostate-specific antigen (PSA) is a key androgen-regulated gene, and is a sensitive and selective marker for prostate cancer screening and assessment (14). Consequently, PSA is used as an indicator of disease progression and response for prostate cancer therapies.

Herein the androgen-dependent LNCaP human prostate cancer cell line (15) was used as a cell model to study the potential mechanisms of VES to prevent prostate cancer development and progression. VES decreases intracellular and secreted levels of PSA in LNCaP cells, which have been cultured either in normal serum or in androgen-stimulated conditions. Furthermore, the results indicate that inhibition of PSA is concomitant with VES-mediated down-regulation of AR protein levels. The inhibition of AR protein is not only because of regulation of AR mRNA level, but also because VES affects the efficiency of AR protein translation.

The LNCaP cell line is derived from lymph node prostate cancer metastasis (15), and is one of the best in vitro models for human prostate cancer studies, as it represents a hormone-refractory prostate carcinoma, and its growth is responsive to androgen. In addition, LNCaP cells express a functional mutant AR, and produce PSA, which is a sensitive and specific tumor marker for prostate cancer screening and assessment (22, 30-32). Whereas both the wild-type AR and the LNCaP mutant respond to androgen, estrogenic compounds and some androgens bind to the LNCaP mutant AR with higher affinity, and more effectively stimulate AR-transcriptional activity and PSA expression (12, 33).

Recently, a 46-kDa tocopherol-associated protein (TAP) has been identified from the cytosol of bovine liver (35). In the followup study, a human TAP (hTAP) was isolated, and the recombinant hTAP was capable of binding to vitamin E with a K_{d} of 460 nM. Northern blotting assays indicate that higher levels of hTAP niRNA are found in the liver, brain, and prostate.

Disclosed herein, unlike other natural products that also showed an inhibitory effect on AR expression at either the transcription (43) or nuclear translocation level (44), vitaminE inhibits the translation of AR. Anti-proliferative therapies can be enhanced by providing reagents that target different pathways or mechanisms for cellular survival or phenotype. Thus, combinations of vitamin E succinate derivatives with other reagents for the treatment or prevention of prostate cancer are disclosed.

1. Vitamin E

The structure of Vitamin E is shown in formula I.

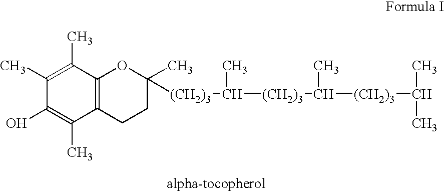

Vitamin E has been shown to be involved in fertility and reproduction. Deficiency of vitamin E in rats leads to absorption in the female and loss of fertility on the male. Vitamin E has been shown to have antioxidant effects, which can protect cells from oxygen and free radical damage. Vitamin E has also been shown to be involved in the formation of red blood cells. Vitamin E can be found in vegetable lipids and in the body fat of animals, but animals cannot produce vitamin E on their own. For example, vitamin E can be found in vegetable oils, nuts and nut oils seeds, egg yolk, margarine, Parmesan, Cheddar, chickpeas, soya beans, wheat germ, oatmeal, avocados, olives, carrots, parsnips, red peppers, green leafy vegetables, sweet potatoes, tomatoes, sweet corn, and watercress.

Vitamin E has a general structure related to the tocopherols, and vitamin E derivatives are typically methylated forms of tocol. There are at least four derivatives of vitamin E which can be naturally: alpha—tocopherol, C29H50 O2 is 5,7,8,-trimethyltocol—which is associated with the strongest general vitamin E activity, beta—tocopherol C28H48 O2 is 5,8,-trimethyltocol, gamma—tocopherol C28H48 O2 is 7,8,-trimethyltocol, and delta—tocopherol C27H46 O2 is 8,-trimethyltocol.

Vitamin E can be found in natural and synthetic forms. The natural forms of vitamin E are typically all of the d-stereoisomer form (RRR-) (for example, d-tocopherol) while the synthetic forms are of the dl variety (for example, dl-tocopherol)

In addition there are variety of esterified derivatives of vitamin E, such as succinate derivative (VES). Esterified derivatives of vitamin E can occur at the ring hydroxyl shown in Formula I. Thus, for example, succinate or acetate can be used to esterify this ring hydroxyl. Another known derivative is RRR-α-tocopheryl succinate. The structure of vitamin E succinate is shown below.

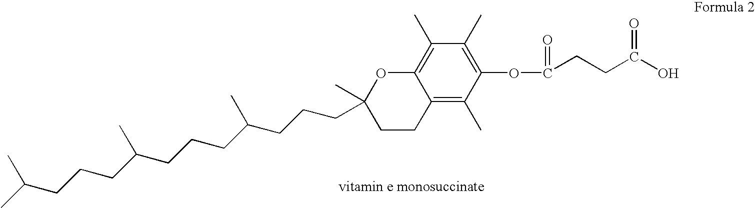

There are a number of different types of prostate cancer therapies. For example, hormonal secretion from the hypothalamus can be modulated by LH-RH agonists, such as Lupron (Formula 3, Cas Nr 0053714-56-0)

5′oxo-Pro-His-Trp-Ser-Tyr-Dleu-Leu-Arg-Pro-NH-CH₂-CH₃

and Zoladex, (Formula 4, Cas Nr. 0065807-02-5)

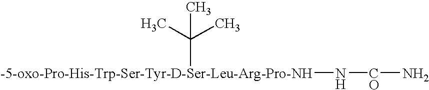

which inhibit the production of T by the testes and adrenal glands. There are also anti-androgen therapeutics, such as Flutamide (Formula 5, 0013311-84-7)

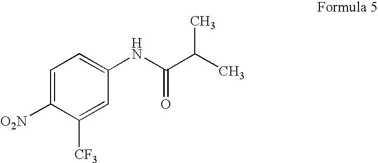

, Casodex (Formula 6, Cas Nr. 0090357-06-5)

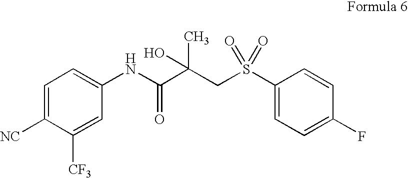

, and Nilutamide (Formula 7, Cas Nr. 0063612-50-0)

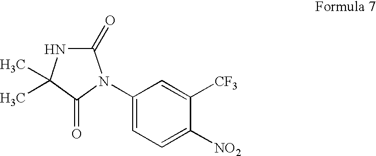

, which can block the androgen binding to AR. Other therapies include the administration of 5-α reductase inhibitors, such as Proscar (Finasteride) (Formula 8 as Nr. 0098319-26-7)

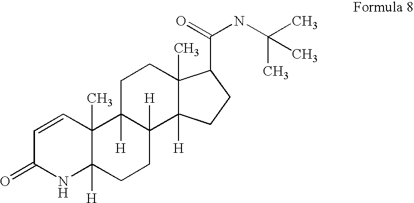

, which can inhibit the conversion of T to DHT. DHT is the most effective ligand for AR with higher binding affinity that T. However, this compound is generally applied for BPH patients than for prostate cancer patients.

Estrogen, such as DES, estradiol, and Stilphosterol Honvan, have also been used in the treatment of prostate cancer. These molecules can decrease the amount of hormones from the hypothalamus. These molecules can decrease the T synthesis from testis by inducing a negative feed-back regulatioin in LH secretion from the pituitary gland and GnRH secretion from the hypothalamus. Other therapeutics include Ketoconazole (Nizoral), which can inhibit the cytochrome p459 enzyme system to reduce T synthesis, and steriods such as Hydrocortisone, Aminoglutethemide (Cytadren), dexmethasome (Decadron), and Cyproterone (Androcur). Ketoconazole is usually used as a second line hormone therapy in patients with stage IV recurrent prostatic cancer. Aminoglutethimide (Cytadren) blocks adrenal steroidogenesis by inhibiting the enzymatic conversion of cholesterol to pregnenolone. Cypoterone is a steroidal antiandrogen with weak progestational activity that results in the partial suppression of pituitary gonadotropin and a decrease in serum T. The main purpose of using Hydrocortisone and Decadron is to relieve the symptoms and increase the quality of life of prostate cancer patients. It is understood that combinations of these therapeutics are performed and herein disclosed.

Thus, disclosed are anti-prostate cancer compounds, such as, flutamide/HF, casodex, niflutamide, finasteride, 1, 25-dihydroxyl, vitamin D3, and natural products including quercetin, resveratrol, silymarin, isoflavonoids, epigallocatechin gallate (EGCG), docosahexaenoic acid (DHA) and eicosapentaenoic acid (EPA). These and others, can all be added in combination with the disclosed vitamin E derivatives, such as VES, collectively or individually in any combination.

Typically, the anti-prostate cancer compounds can be provided at concentrations of less than or equal to 20 uM, 15 uM, 10, uM, 5 uM, 2 uM, 1 uM, 0.1 uM, or 0.01 uM. Typically the anti-androgens can also be provided at concentrations of less than or equal to 20 uM, 15 uM, 10, uM, 5 uM, 2 uM, 1 uM, 0.1 uM, or 0.01 uM. Typically the vitamin E derivatives, such as VES, can be administered at Typically the anti-androgens can also be provided at concentrations of less than or equal to 100 uM, 90 uM, 80 uM, 70 uM, 60 uM, 50 uM, 40 uM, 30 uM, 20 uM, 15 uM, 10, uM, S uM, 2 uM, 1 uM, 0.1 uM, or 0.01 uM. However those of skill in the art understand how to assay for the optimal concentration for administration in vivo, of any of the disclosed compositions, by for example, relying on disclosed cell and animal models for action, as well as by testing the compositions in vivo at various concentrations.

B. Methods of Making the Compositions

The compositions can be made using the methods disclosed herein or by any method known to one of skill in the art. The compositions can also be purchased from for example, Sigma Inc.

C. Methods of Using the Compositions

The disclosed compositions can be used to reduce the proliferation of prostate cancer cells. Thus, these compositions can be used in therapies directed at prostate cancer, and they can be used in conjunction with other prostate cancer therapies, such as the administration of anti-androgens, such as hydroxyflutamide (HF).

1. Delivery of Pharamceutical Products

A composition is a pharmaceutical composition is a composition appropriately formulated such that it can be administered to a subject Typically this would mean that the composition is present with a pharmaceutically acceptable carrier as discussed herein. As described above, the compositions can also be administered in vivo in a pharmaceutically acceptable carrier. By “pharmaceutically acceptable” is meant a material that is not biologically or otherwise undesirable, i.e., the material may be administered to a subject, along with the nucleic acid or vector, without causing any undesirable biological effects or interacting in a deleterious manner with any of the other components of the pharmaceutical composition in which it is contained. The carrier would naturally be selected to minimize any degradation of the active ingredient(s) and to minimize any adverse side effects in the subject, as would be well known to one of skill in the art.

The compositions may be administered orally, parenterally (e.g., intravenously), by intramuscular injection, by intraperitoneal injection, transdermally, extracorporeally, topically or the like, although topical intranasal administration or administration by inhalant is typically preferred. As used herein, “topical intranasal administration” means delivery of the compositions into the nose and nasal passages through one or both of the nares and can comprise delivery by a spraying mechanism or droplet mechanism, or through aerosolization of the nucleic acid or vector. The latter may be effective when a large number of animals, such as humans, are to be treated simultaneously. Administration of the compositions by inhalant can be through the nose or mouth via delivery by a spraying or droplet mechanism. Delivery can also be directly to any area of the respiratory system (e.g., lungs) via intubation. The exact amount of the compositions required will vary from subject to subject, depending on the species, age, weight and general condition of the subject, the severity of the allergic disorder being treated, the particular nucleic acid or vector used, its mode of administration and the like. Thus, it is not possible to specify an exact amount for every composition. However, an appropriate amount can be determined by one of ordinary skill in the art using only routine experimentation given the teachings herein.

Parenteral administration of the composition, if used, is generally characterized by injection. Injectables can be prepared in conventional forms, either as liquid solutions or suspensions, solid forms suitable for solution of suspension in liquid prior to injection, or as emulsions. A more recently revised approach for parenteral administration involves use of a slow release or sustained release system such that a constant dosage is maintained. See, e.g., U.S. Pat. No. 3,610,795, which is incorporated by reference herein.

The materials may be in solution, suspension (for example, incorporated into microparticles, liposomes, or cells). These may be targeted to a particular cell type via antibodies, receptors, or receptor ligands. The following references are examples of the use of this technology to target specific proteins to tumor tissue (Senter, et al., Bioconjugate Chem., 2:447-451, (1991); Bagshawe, K. D., Br. J. Cancer, 60:275-281, (1989); Bagshawe, et al., Br. J. Cancer, 58:700-703, (1988); Senter, et al., Bioconjugate Chem., 4:3-9, (1993); Battelli, et al., Cancer Immunol. Immunother., 35:421-425, (1992); Pietersz and McKenzie, Immunolog. Reviews, 129:57-80, (1992); and Roffler, et al., Biochem. Pharmacol, 42:2062-2065, (1991)). Vehicles such as “stealth” and other antibody conjugated liposomes (including lipid mediated drug targeting to colonic carcinoma), receptor mediated targeting of DNA through cell specific ligands, lymphocyte directed tumor targeting, and highly specific therapeutic retroviral targeting of murine glioma cells in vivo. The following references are examples of the use of this technology to target specific proteins to tumor tissue (Hughes et al., Cancer Research, 49:6214-6220, (1989); and Litzinger and Huang, Biochimica et Biophysica Acta, 1104:179-187, (1992)). In general, receptors are involved in pathways of endocytosis, either constitutive or ligand induced. These receptors cluster in clathrin-coated pits, enter the cell via clathrin-coated vesicles, pass through an acidified endosome in which the receptors are sorted, and then either recycle to the cell surface, become stored intracellularly, or are degraded in lysosomes. The internalization pathways serve a variety of functions, such as nutrient uptake, removal of activated proteins, clearance of macromolecules, opportunistic entry of viruses and toxins, dissociation and degradation of ligand, and receptor-level regulation. Many receptors follow more than one intracellular pathway, depending on the cell type, receptor concentration, type of ligand, ligand valency, and ligand concentration. Molecular and cellular mechanisms of receptor-mediated endocytosis has been reviewed (Brown and Greene, DNA and Cell Biology 10:6, 399-409 (1991)).

a) Pharmaceutically Acceptable Carriers

The compositions can be used therapeutically in combination with a pharmaceutically acceptable carrier.

Pharmaceutical carriers are known to those skilled in the art. These most typically would be standard carriers for administration of drugs to humans, including solutions such as sterile water, saline, and buffered solutions at physiological pH. The compositions can be administered intramuscularly or subcutaneously. Any compound or composition that allows for the delivery of another composition to a subject, such that the delivery itself is not detrimental to the subject can be considered pharmaceutically acceptable carrier. Other compounds will be administered according to standard procedures used by those skilled in the art.

Pharmaceutical compositions may include carriers, thickeners, diluents, buffers, preservatives, surface active agents and the like in addition to the molecule of choice. Pharmaceutical compositions may also include one or more active ingredients such as antimicrobial agents, anti-inflammatory agents, anesthetics, and the like.

The pharmaceutical composition may be administered in a number of ways depending on whether local or systemic treatment is desired, and on the area to be treated. Administration may be topically (including ophthalmically, vaginally, rectally, intranasally), orally, by inhalation, or parenterally, for example by intravenous drip, subcutaneous, intraperitoneal or intramuscular injection. The disclosed compositions can be administered intravenously, intraperitoneally, intramuscularly, subcutaneously, intracavity, or transdermally.

Preparations for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for topical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids or binders may be desirable.

Some of the compositions may potentially be administered as a pharmaceutically acceptable acid- or base- addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mono-, di-, trialkyl and aryl amines and substituted ethanolamines.

b) Therapeutic Uses

The dosage ranges for the administration of the compositions are those large enough to produce the desired effect in which the symptoms disorder are affected. The dosage should not be so large as to cause adverse side effects, such as unwanted cross-reactions, anaphylactic reactions, and the like. Generally, the dosage will vary with the age, condition, sex and extent of the disease in the patient and can be determined by one of skill in the art. The dosage can be adjusted by the individual physician in the event of any counterindications. Dosage can vary, and can be administered in one or more dose administrations daily, for one or several days.

Preferred are pharmaceutical compositions comprising VES or VES derivatives that inhibit PSA and antiandrogen compounds that inhibit androgen activity. A preferred antiandrogen compound is HF.

The disclosed compositions can also be used as standards in a LNCaP cell growth assay to determine the efficacy of putative prostate cancer therapeutics.

### D. EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1

Epidemiological evidence indicates that a daily supplement with vitamin E could reduce the risk of prostate cancer. Disclosed herein, vitamin E succinate (α-VES) suppressed the expression of prostate-specific antigen (PSA), a marker for the progression of prostate cancer. α-VES suppressed androgen receptor (AR) expression via post-transcription modulation-protein degradation. Cell growth studies and the MTT assay further showed α-VES can inhibit the growth of prostate cancer LNCaP cells. In contrast, hydroxyflutamide (HF), an anti-androgen currently used for treatment of prostate cancer patients, showed only marginal inhibition for LNCaP cell growth. Interestingly, simultaneous addition of HF and α-VES results in a more significant inhibition in the LNCaP cells growth. Compared with α-Vit E and r-Vit E, these results also suggested that the VES is the most effective compound to inhibit the LNCaP cell growth. Together, this data indicated a relationship between α-VES degradation of androgen/AR and androgen/AR-mediated cell growth. This type of mechanism provides new opportunities for therapeutics directed towards prostate cancer.

a) Materials and Methods

(1) Chemicals and Reagents

RRR-α-tocopheryl succinate, (+)-γ-tocopherol, succinic acid and 5-dihydrotestosterone (DHT) were purchased from Sigma Chemical Co. (St. Louis, Mo.), 1α, 25-dihydroxyvitamin D₃ (Vit D₃) was purchased from Fluka, hydroxyflutamide (HF) was a gift from Schering. EXPRE35S35S protein labeling mixture was purchased from NEN Life Science Products Inc (Boston, Mass.). VES, succinic acid (Suc), selenomethionine (SM), and 5-dihydrotestosterone (DHT) were purchased from Sigma. Antibodies to vitamin D receptor (VDR), peroxisome-proliferator activated receptor (PPAR), and retinoid X receptor (RXR) and—actin were from Santa Cruz Biotechnology. PSA (clone ER-PR8) antibody was purchased from Dako.

(2) Cell Culture and VES Treatment

The human prostate cancer cell line LNCaP was obtained from the American Type Culture collection (Rockville, Md.). Fibroblast cell was primarily cultured from normal prostate tissue. LNCaP cells were propagated in 12-well, 60 mm or 100 mm culture dishes at the desired density in RPMI 1640 medium (Gibco, Rockville, Md.) supplemented with 8% fetal bovine serum (FBS) (Gibco, Rockville, Md.) at 37° C. and 5% CO₂ until reaching 60%-80% confluence or were grown in phenol red-free RPMI medium 1640 with 8% fetal bovine serum (FBS). The fibroblast cells were maintained in DMEM (Gibco, Rockville, Md.) with 10% FBS. The cells were treated with VES at designated concentrations with or without other compounds (HF, Vit D₃, DHT, Succinic acid). The cells were treated with Suc as a control, VES, HF, SM, or DHT at designated concentrations. During the treatment, the medium was changed every 4 days and fresh compounds were added every 2 days.

(3) Cell Counting and Thiazolyl Blue (MTT) Assay

The MTT assay is a quantitative colorimetric assay for mammalian cell survival and proliferation (13, 16). The 5×10⁴ LNCaP cells were seeded in 12-well plates. After 36-48 h, the medium was changed to phenol red free RPMI1640 with 8 % FBS or CS-FBS for another 2, 4, and 6 days, with different compound treatment with or without ligand. At the end of each time period treatment, 200 μl of MTT (5 mg/ml; Sigma) was added into the each well with 1 ml of medium for 3 h at 37° C. After incubation, 2 ml of 0.04 M HCl in isopropyl alcohol was added into each well. After 30-min shaking at room temperature and several pippettings, the absorbency was read at a test wavelength of 595 nm. The 2×10⁵ fibroblast cells were seeded in each well of 6-well dishes. The treatment and MTT assay were similar as LNCaP cell. For cell counting, cells were trypsinized, neutralized by medium, and counted on hemocytometers. Fibroblast cells were seeded at 2×105 per well in 6-well plates, and cell growth assays were conducted by using the same MTT assay used for LNCaP cells.

(4) Western Blotting

Total protein lysate from LNCaP cells was prepared as previously described (Yeh, PNAS). After separation of 50 μg protein by SDS-PAGE gel, proteins were transferred by electrophoresis to Immobilon-P membrane (Millipore Corp., Bedford, Mass.) and incubated in PBS with 0.1% Tween-20 and 10% FCS for 2 h. Primary antibodies specific for human AR, PSA, and β-actin were diluted in PBS with 0.1% Tween-20 (PBST) as described in manual and incubated at room temperature for 2 h. Membranes were washed in PBST (three times, 10 min each time) and incubated with AP-conjugated secondary antibody which was diluted as described in manual in PBST and incubated for 2 h in room temperature, washed in PBST (three times, 10 min each time). The proteins were detected by AP western blotting reagents.

(5) Northern Blot Analysis

LNCaP cell was treated with designed concentration of VES or other compound. Cells were harvest after 1, 2, and 3 days treatment. Total RNA was extracted using Trizol, according to manufacturer's directions (GIBCO). Total RNA (20 μg) was electrophoresed though formaldehyde-agarose gels and transferred to Hybond-N+ membrane (Amersham Pharmacia Biotech) according the company's protocol (17).

For Northern blots, the fragments of the human PSA, AR, and β-actin cDNA were labeled with [³²P]-dCTP using the random primed DNA labeling kit from Amersham Pharmacia Biotech. Membranes were prehybridized, hybridized, and washed using Rapid-hyb system from Amersham Pharmacia Biotech, according to the manufacturer's user manual. The mRNA was detected using phosphorimager screen system (Molecular Dynamics).

(6) [³⁵S]-Methionine Labeling of AR in LNCaP Cells

LNCaP cells were plated into 100-mm dishes and grown for 72-96 h till to 80% confluence. All pulse media, chase media, and wash media used in the following procedures were warmed to 37° C. before use. Cells were washed with pre-warmed PBS once between medium changes with methionine-free DMEM+1% penicillin-streptomycin and 5% dialyzed fetal calf serum for 2 h at 37° C. At the same time, LNCaP cells were pretreated by 10 μM VES or ethanol(0.1% vol/vol) typically for 24 hours. All media were added to cells at a volume of 5 ml/dish. After 2 h, cells were labeled by incubation with pulse medium either 1) for 1 h followed by 2, 6, and 12 h incubation with chase medium for protein stability assay or 2) for 0.5, 2, 6, and 12 h with no chase period for protein translation assay. Pulse medium consisted of 100 μCi/ml [35S]methionine (1 Ci=37 GBq) and 5 μM unlabeled methionine in methionine-free DMEM with 5% dialyzed FBS. To lyse cells, precooled RIPA buffer (1% Nonidet P-40/0.1% SDS/0.5% sodium deoxycholate/1×PBS) plus 1 mM PMSF was added to each dish. Before harvested, LNCaP cells were washed with pre-warmed PBS twice.

(7) Lysis of [³⁵S]-methionine Labeled Cells

To lyse cells, 0.6 ml pre-cooled RIPA buffer plus 1 mM PMSF was added to each dish, which was placed on ice for 2 min. The solution was transferred to microcentrifuge tubes, and cellular debris pelleted by centrifugation at 10,000 rpm for 5 minutes at 4° C. Supernatant was transferred to a new microcentrifuge tube and stored in −70° C. until immunoprecipitation was performed.

(8) Immunoprecipitation of [³⁵S]-Methionine Labeled Cell Lysate by AntiAR Antibody—

Three hundred micrograms of total cellular protein was transferred to new microcentrifuge tubes, and then 3 μl of rabbit anti-AR polyclonal antibody-NH27 (19) and 500 μl of reaction buffer (0.15 NaCl/0% Triton X-100/20 mM TrisHCl, pH 8.0) was added (20), and incubated for 2 h at 4° C. with constant rocking. Twenty-five microliters of protein A/G beads, was added to the solution and incubated for 2 h at 4° C. with constant rocking. Samples were centrifuged at 2,500×g for 3 min at 4° C. to collect the beads and then washed three times using ice-cold reaction buffer. Fifty microliters of 1.5×SDS gel-loading buffer was added and boiled for 4 min. Aliquots (25 μl)were subjected to gel electrophoresis, followed by autoradiographic signal quantitation using IQMAC software (Molecular Dynamics).

(9) Cell Transfection and Reporter Gene Assay.

For PSA promoter luciferase assay, LNCaP cells were plated in 60-mm dishes until 60-70% confluence, and then transfected with 6-kb PSA promoter-linked luciferase reporter (PSA6.0-Luc) by using Superfect (Qiagen, Valencia, Calif.). Twenty-four hours after transfection, the cells were treated with various compounds for an additional 24 h. For AR N-terminal/C-terminal (N-C) interaction assay, COS-1 cells (1×105) were plated on 12-well plates 12 h before being transfected with 0.5 μg of pG5-Luc reporter and other expression vectors depicted in the figure legends. After 24 h transfection, 10 nM DHT and/or 10 μM VES was added for another 24 h. For each transfection, simian virus 40 promoter driven Renilla luciferase (SV40RL) was used as an internal control.

(10) In Vivo AR Radioligand Competition Binding Assay.

LNCaP cells were plated into 60-mm dishes and grown to 60% confluence. Cells were pretreated with ethanol or 10 μM VES (0.1% vol/vol) for 24 h. Then medium was changed to RPMI 1640 with 8% CS-FBS, and competition ligand binding was performed by using 2.5 nM [3H]R1881, with or without 100-fold excess of unlabeled R1881 (250 nM) (18). After 1-h incubation, cells were harvested by lysis buffer (PBS with 1% Triton X-100). Equal protein amounts of cell extract were subjected to binding assays, which were terminated by adding hydroxylapatite. Each sample was filtered by using a sampling manifold (Millipore) and unbound ligand was removed by washing. Filter papers that contained bound ligand were transferred to counting vials containing 5 ml of liquid scintillation fluid and counted with a multipurpose scintillation counter (Beckman).

b) Results

(1) VES Represses the Growth of LNCaP Cells, but Not Prostate Fibroblasts.

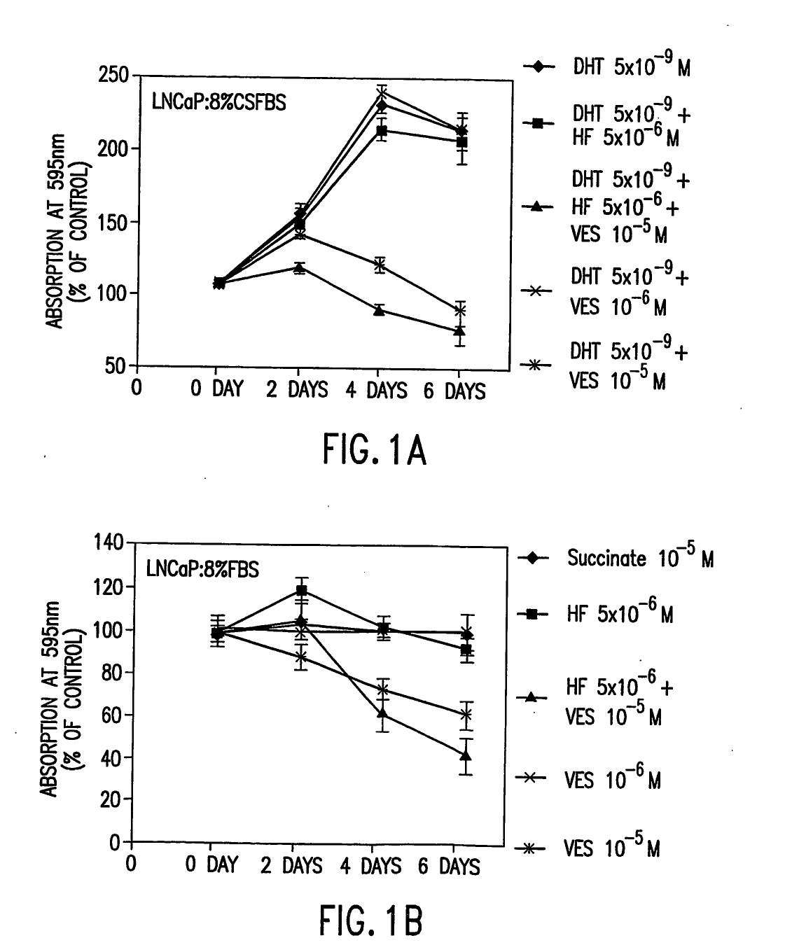

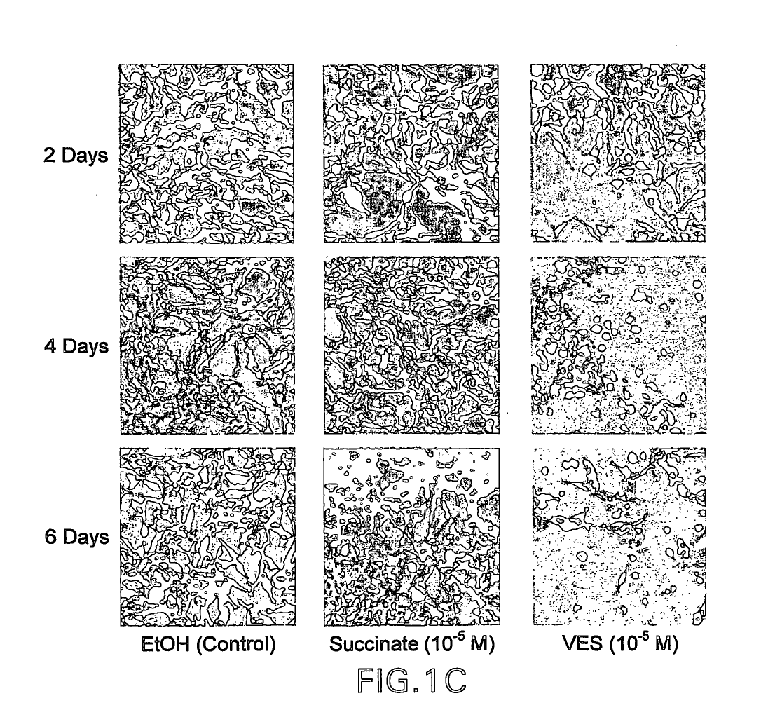

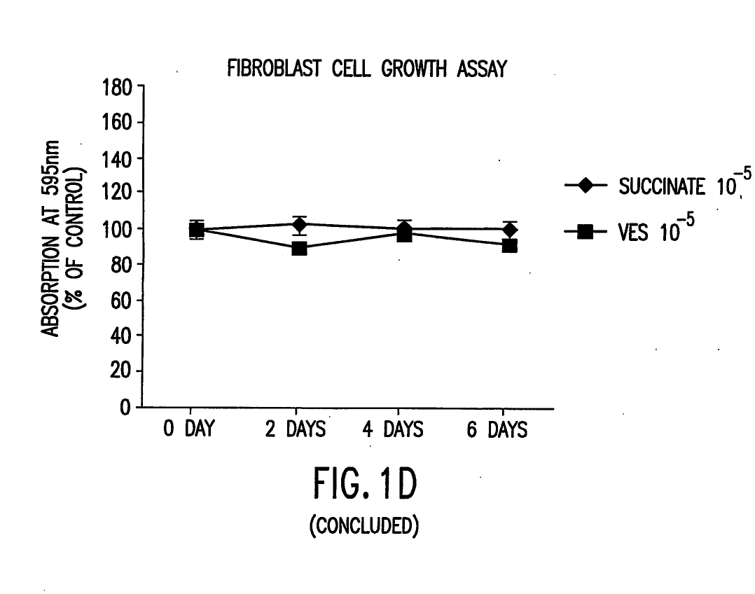

Many prostate tumors progress to a hormone-refractory stage concomitant with the flutamide withdrawal syndrome (21), enabling the tumor to grow in the presence of antiandrogens, such as HF. It is necessary, therefore, to search for more effective antiproliferative reagents to manage prostate cancer. Here, the inhibitory effect of VES with HF in LNCaP cells was compared. Using the MTT assay, FIG. 1A demonstrates that 5nM DHT can stimulate LNCaP cell growth, and the addition of 5 μM HF fails to repress this DHT-induced cell growth in medium with 8% CS-FBS. In contrast, the addition of 10 μM VES effectively represses DHT-mediated cell growth. Addition of both 5 μM HF and 10 μM VES can further repress DHT-mediated cell growth. In addition, when 8% CS-FBS was replaced with 8% FBS without DHT, 5 μM HF induces LNCaP cell growth at day 2, with the induction gradually diminishing after day 4 (FIG. 1B). Again, 10 μM VES inhibits LNCaP cell growth and the combination of both 10 μM VES and 5 μM HF could further repress LNCaP cell growth after day 4. Together, results from FIGS. 1A and B demonstrate that 10 μM VES can effectively inhibit LNCaP cell growth, either in FBS or in CS-FBS in the presence of 5 nM DHT. The combination of 10 μM VES and 5 μM HF further represses LNCaP cell growth. At the same time, a morphologic change was observed in the LNCaP cells during the treatment period with most of the cells dying after VES treatment for 4 days (FIG. 1C).

When tumor cells were replaced with primary cultured fibroblasts from normal prostate tissue, 10 μM VES had only a marginal inhibitory effect on cell growth (FIG. 1D), suggesting that VES may have selective inhibitory effects on tumor cells that are androgen sensitive. Direct cell-number counting by using a hemocytometer further confirmed these cell growth results.

(2) VES Inhibits the Expression of PSA.

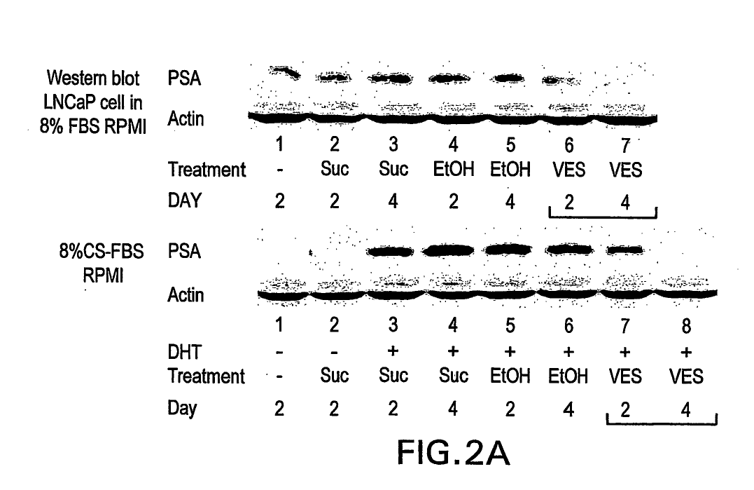

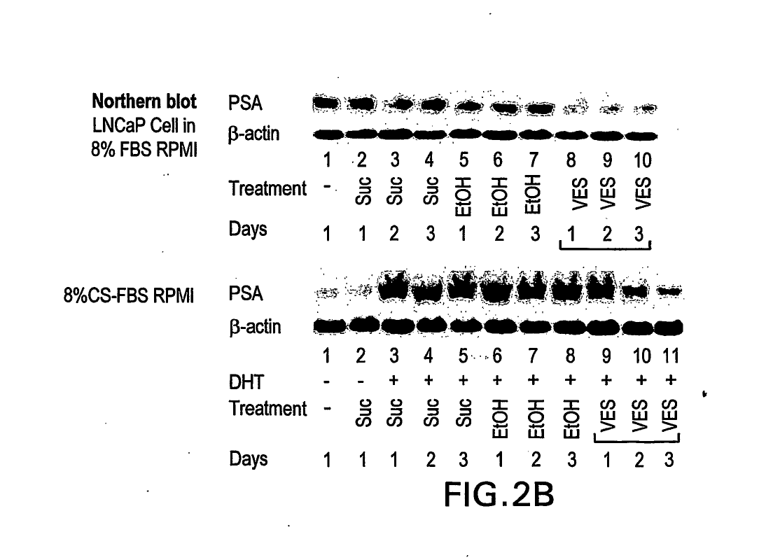

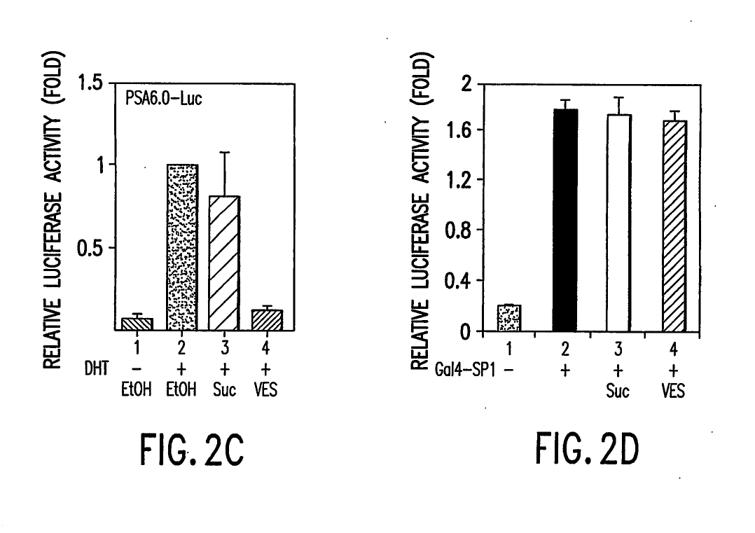

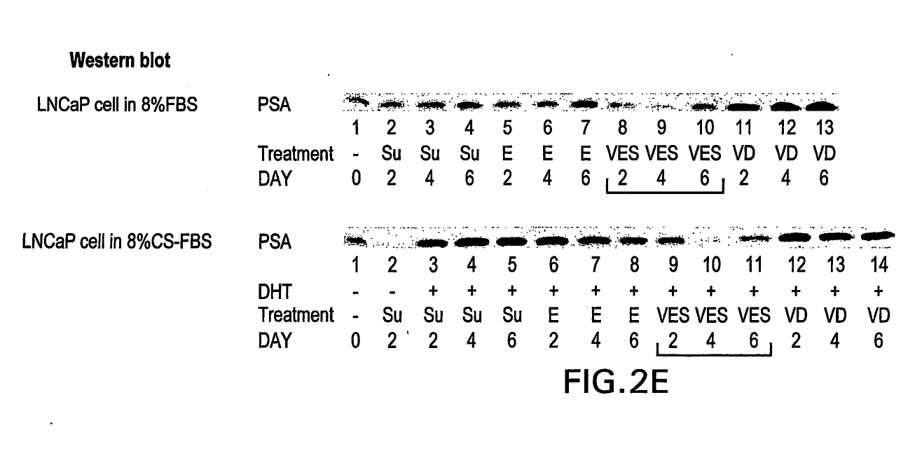

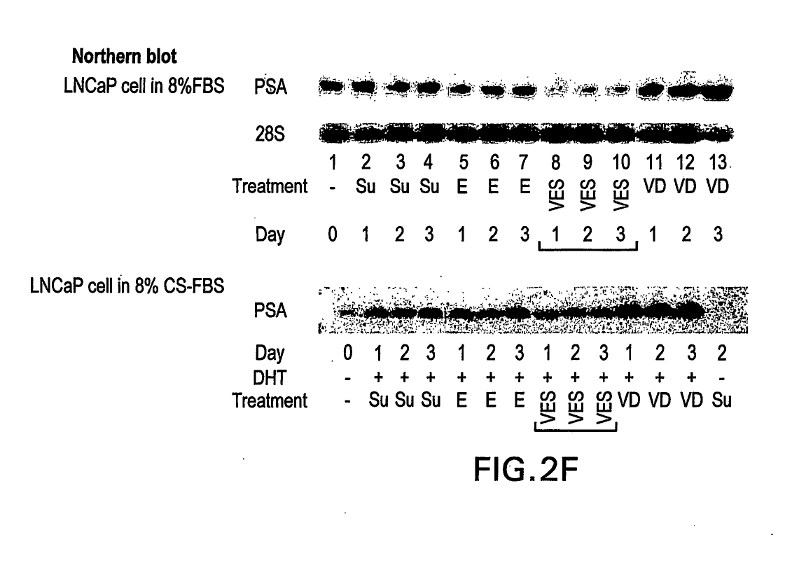

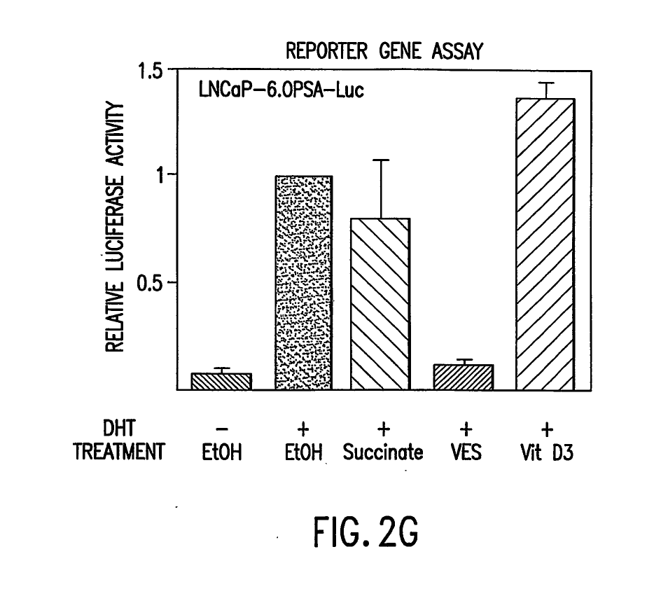

As shown in FIGS. 2A and B, using Western blotting and Northern blotting analyses, it was found that both mRNA and protein expression of PSA were induced by 5 nM DHT, and the addition of 10 μM VES effectively repressed PSA expression at both the mRNA and protein levels in LNCaP cells cultured under the same conditions as described for FIGS. 1A and B. To further study whether VES-repressed PSA expression occurred at the transcription level, a luciferase reporter linked with the 6.0-kbPSA promoter (PSA6.0-Luc) was used to assay the VES effect. As shown in FIG. 2C, 5 nM DHT induced PSA6.0-Luc activity, and the addition of 10 μM VES, but not Suc, repressed DHT induced-PSA6.0-Luc activity. To test whether the VES-mediated inhibition of PSA promoter is specific, the effect of VES on the transactivation of SP1 was examined by testing GAL4 DNA-binding domain (DBD) fused SP1, which can bind to and activate GALA binding site-linked luciferase reporter, pG5-Luc. The results indicate that 10 μM VES did not significantly inhibit GAL4-SP1 transcription activity (FIG. 2D). Together, our data show that 10 μM VES not only represses DHT-mediated cell growth, but also selectively represses DHT-induced PSA expression in LNCaP cells.

(3) VES Affects AR mRNA and Protein Expression.

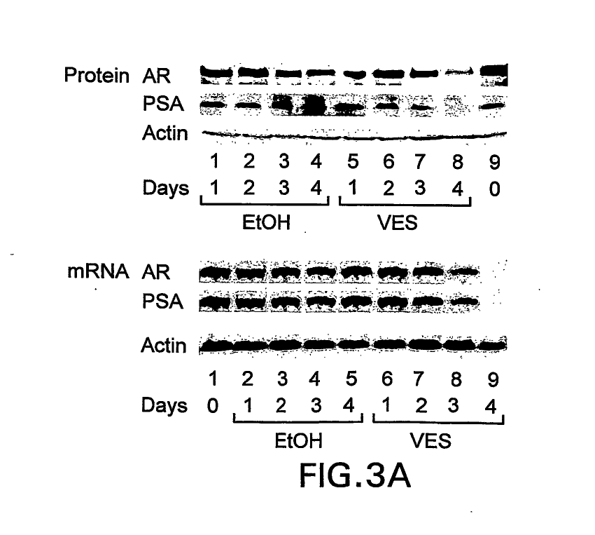

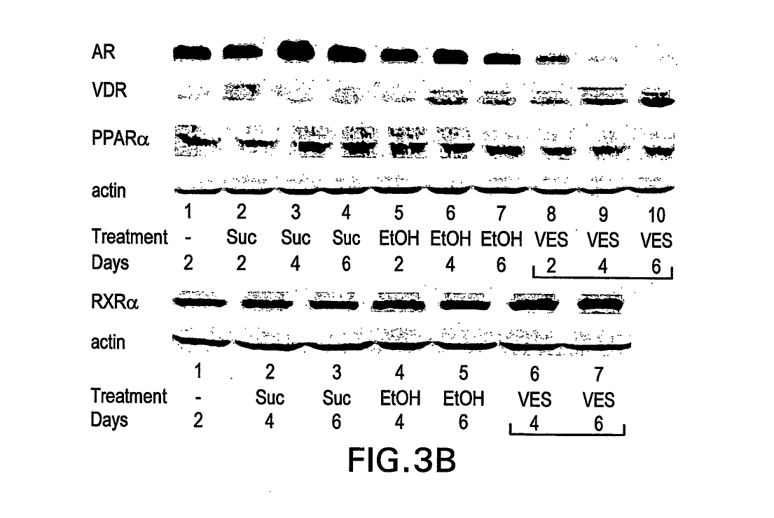

As shown in FIG. 3A, Northern blotting data indicate that VES inhibits AR mRNA and protein expression; however, PSA mRNA and protein levels begin to decrease at earlier times. Next, to determine whether VES affects the AR at protein level, LNCaP cells were cultured in RPMI 8% FCS or CS-FCS with 5 nM DHT in the absence or presence of 10 uM VES. Whole cell extracts were collected for Western blotting analyses. Using NH27 anti-AR antibody, the results indicated that 10 uM VES but not 10 nM Vit D could suppress AR expression at the protein level. This repression is specific as 10 uM VES showed little effect on the PPARr expression (FIGS. 2e-f).

(4) VES Does Not Affect the Ligand-Binding, N-C Dimerization, or Nuclear Translocation of AR.

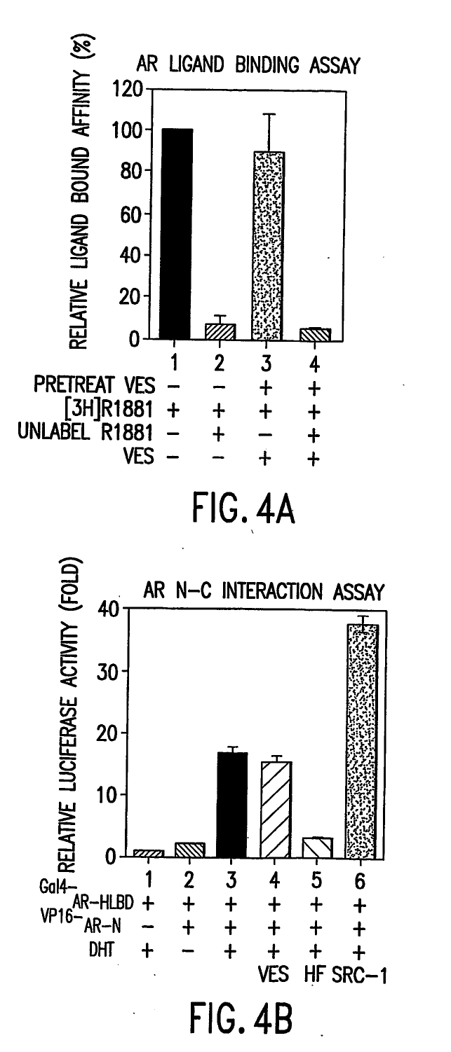

After binding to androgen(s), AR will form a dimer (23), translocate from the cytoplasm to the nucleus (24), and activate its target genes by recognition of androgen-response elements (25). First, a competition radioligand-binding assay was used to examine whether VES would affect AR-ligand-binding ability. Results show that unlabeled R1881 can compete for 95% of the specific binding, and VES treatment has little influence on AR ligand binding (FIG. 4A). Next, whether VES affects the N-C interaction of AR, which has been suggested to play an important role in AR transactivation (26) was examined. A mammalian two-hybrid system, which included the hinge and ligand-binding domain of AR fused with the GAL4-DBD (GAL-ARHLBD), the N terminus of AR fused with VP16 (VP16-ARN), and a pG5-Luc reporter (23) was used. The results show that 10 nM DHT triggers the AR N-C interaction and addition of 10 μM VES has little influence on the AR N-C interaction (FIG. 4B, lane 3 vs. 4). Whether VES could influence translocation of AR was examined. Although VES has little influence on the AR distribution between cytosol and nucleus, the total AR-staining intensity is reduced, suggesting that VES may affect AR protein expression. These immunostaining results not only confirm the Northern and Western blotting assays, but also indicate that VES may function via a posttranscription pathway to down-regulate AR protein function.

Together, the data indicate that VES cannot influence the ligand-binding, N-C dimerization, and nuclear translocation of AR. Instead, VES reduces the overall AR-staining intensity, suggesting that VES may affect AR expression at the transcriptional or translational level.

(5) VES Inhibits AR Protein Translation in LNCaP Cells.

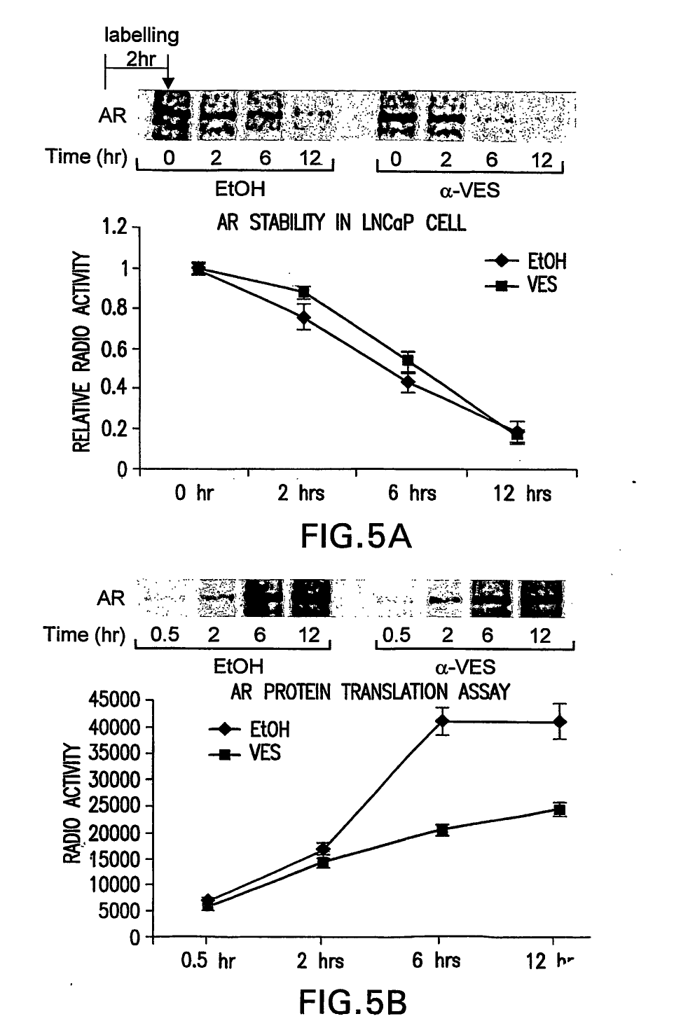

To determine the possible mechanism involved in the regulation of AR expression at the posttranscriptional level, a pulse-chase labeling was applied to characterize whether VES affects AR-protein-translation efficiency or stability (27). Although the intensity of signal is different at the starting point, the degradation rates of the AR are similar in the absence or presence of VES. The data indicate that VES has little effect on AR-protein stability (FIG. 5A). On the other hand, after treatment with VES, AR-protein synthesis is much slower compared with that of the control group (FIG. 5B). These results suggest that VES may regulate AR protein level through inhibition of protein translation rather than influencing stability.

(6) VES Differentially Regulates the Expression of AR, VDR, PPARα, and RXRα.

To test whether the VES-mediated down-regulation of AR function is specific, the expression level of other nuclearreceptors under the same conditions was examined. When antibodies for AR, VDR, PPARα, and RXRα were used, the results indicated that 10 μM VES, but not 10 μM Suc, could suppress AR protein level. This VES-mediated AR repression is selective as 10 μM VES showed little effect on the PPARα and RXRα expression (FIG. 3B) and, in contrast, increased the expression of VDR (FIG. 3B).

(7) VES, but not Selenium, Affects AR and PSA Expression.

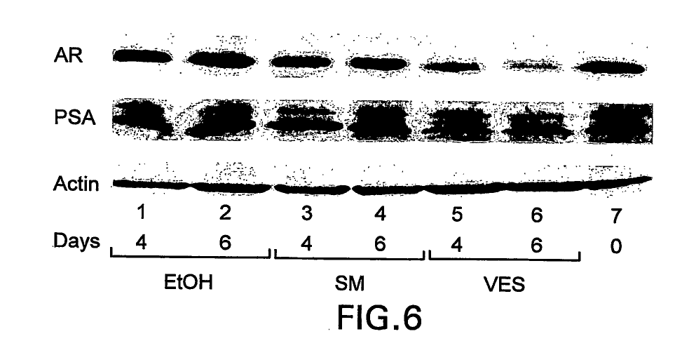

SM is known to be the major source of selenium in the diet. 10 μM SM, which has been reported to inhibit LNCaP cell growth (29), was used. Although the SM-mediated growth inhibition in LNCaP cells after 4 days treatment was observed, Western blot data indicated that SM has no effect on AR and PSA expression (FIG. 6). Together, the results suggest that VES, but not selenium, down-regulates the expression of AR and PSA. The VES-mediated growth inhibition of prostate cancer cells may be partly due to down-regulated AR expression, and SM may function through other mechanisms to inhibit the growth of prostate cancer cells.

(8) Pulse-chase Labeling to Detect the Stability of AR in LNCaP Cells

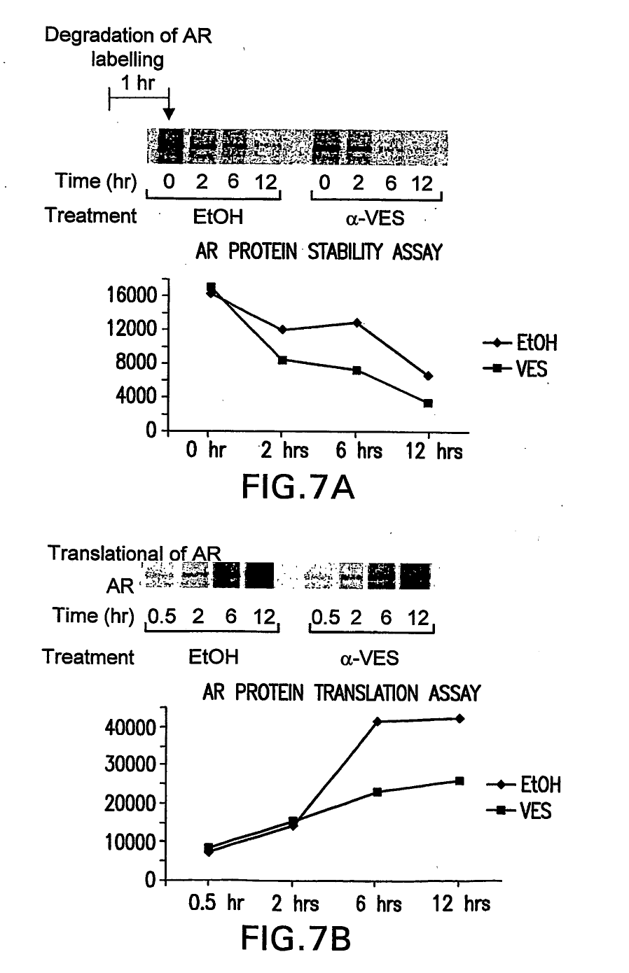

VES effectively affects the protein level of AR expression. Pulse-chase labeling was performed to characterize whether VES affects the protein translational efficiency and stability. As shown in FIG. 7, these results indicated that VES affects the protein stability of AR (from 2 h to 6 h) and consequently inhibit the accumulation of AR protein.

(9) The Effects of α-Vit E, γ-Vit E and VES on the Growth of LNCaP

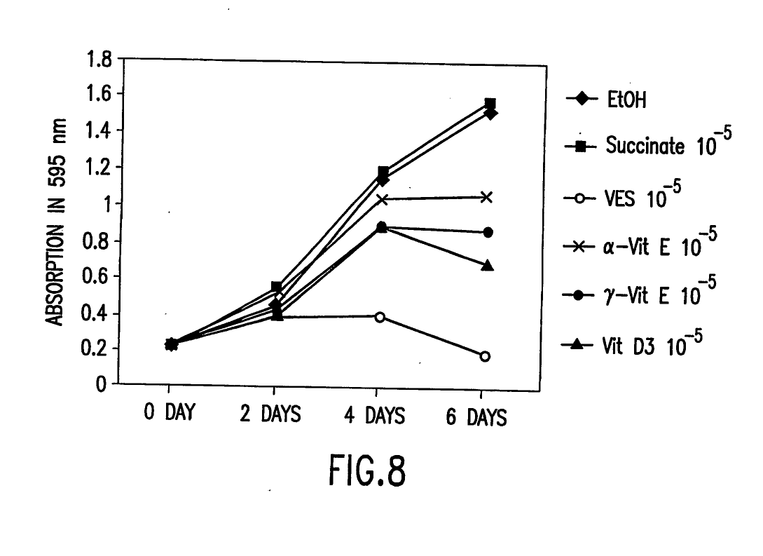

As shown in FIG. 8, VES, Vit D, α-Vit E, and γ-Vit E can inhibit the cell growth in RPMI 8% FBS. However, the VES is the most effective compound, which does not correlate with its anti-oxidant capacity compared with other compounds.

Microarray technology has been applied to further characterize the downstream targets of VES-mediated biological events.

(10) VES Differentially Inhibit the Growth of Cancer Cells

A primary cell culture of fibroblasts was established to show the efficiency of VES-mediated growth effects. These results indicated that VES differentially inhibit the LNCaP, but not primary cultured fibroblast cell growth.

(11) VES, VDR, and Prostate Cancer

Western blotting analysis of AR expression, showed that 10 μM VES can induce VDR expression.

(12) Succinate Derivatives vs. Acetate Derivatives

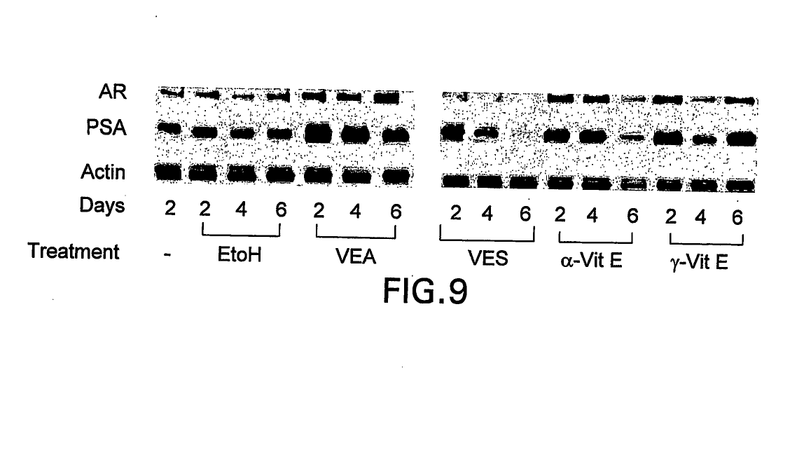

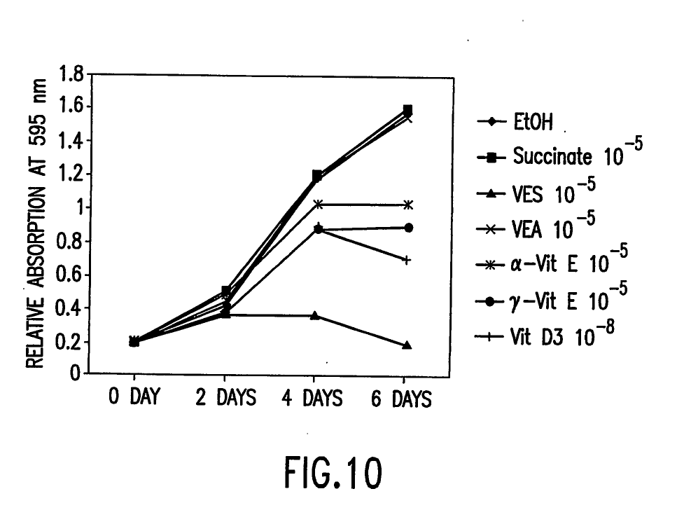

The effects of α-Vit E succinate (VES), α-Vit E acetate (VEA), α-Vit E, and γ-Vit E on the cell growth, and AR and PSA expression, were compared. The results indicated that VES is the most effective isoform of Vit E, and that α-Vit E can also inhibit PSA and AR expression. However, VEA or γ-Vit E do not efficiently inhibit the expression of AR and PSA. Together, among the tested vit E analogs, these results indicate that VES is the most effective isoform for the growth inhibition of prostate cancer cells. (FIGS. 9 and 10)

E. References

1. Traber, M. G. & Packer, L. (1995) Am. J. Clin. Nutr. 62, 1501S-1509S.

2. The Alpha-Tocopherol & Beta Carotene Cancer Prevention Study Group. (1994) N. Engl. J. Med. 330, 1029-1035.

3. Giovannucci, E. (2000) J. Natl. Cancer Inst.92, 1966-1967.

4. Helzlsouer, K. J., Huang, H. Y., Alberg, A. J., Hoffman, S., Burke, A., Norkus, E. P., Morris, J. S. & Comstock, G. W. (2000) J. Natl. Cancer Inst. 92, 2018-2023.

5. Neuzil, J., Weber, T., Schroder, A., Lu, M., Ostermann, G., Gellert, N., Mayne, G. C., Olejnicka, B., Negre-Salvayre, A., Sticha, M., et al. (2001) FASEB J. 15, 403-415.

6. Neuzil, J., Weber, T., Terman, A., Weber, C. & Brunk, U. T. (2001) Redox Rep. 6, 143-151.

7. Israel, K., Sanders, B. G. & Kline, K. (1995) Nutr. Cancer 24, 161-169.

8. Landis, S. H., Murray, T., Bolden, S. & Wingo, P. A. (1999) CA Cancer J. Clin. 49, 8-31.

9. Sogani, P. C. & Whitmore, W. F., Jr. (1988) Cancer Treat. Res. 39, 131-145.

10. Fenton, M. A., Shuster, T. D., Fertig, A. M., Taplin, M. E., Kolvenbag, G., Bubley, G. J. & Balk, S. P. (1997) Clin. Cancer Res. 3, 1383-1388.

11. Yeh, S., Miyamoto, H., Shima, H. & Chang, C. (1998) Proc. Natl. Acad. Sci. USA 95, 5527-5532.

12. Grigoryev, D. N., Long, B. J., Njar, V. C. & Brodie, A. H. (2000) J. Steroid Biochem. Mol. Biol 75, 1-10.

13. Yeh, S., Lin, H. K., Kang, H. Y., Thin, T. H., Lin, M. F. & Chang, C. (1999) Proc. Natl. Acad. Sci. USA 96, 5458-5463.

14. Vernon, S. E. & Williams, W. D. (1983) J Urol. 130, 95-98.

15. Horoszewicz, J. S., Leong, S. S., Kawinski, E., Karr, J. P., Rosenthal, H., Chu, T. M., Mirand, E. A. & Murphy, G. P. (1983) Cancer Res. 43, 1809-1818.

16. Mosmann, T. (1983) J. Immunol. Methods 65, 55-63.

17. Sambrook, J. & Russell, D. W. (2001) Molecular Cloning: A Laboratory Manual (Cold Spring Harbor Lab. Press, Plainview, N.Y.)

18. Ekman, P., Barrack, E. R., Greene, G. L., Jensen, E. V. & Walsh, P. C. (1983) J. Clin. Endocrinol. Metab. 57, 166-176.

19. Lin, H. K., Yeh, S., Kang, H. Y. & Chang, C. (2001) Proc. Natl. Acad. Sci. USA 98, 7200-7205.

20. Yang, C. R., Leskov, K., Hosley-Eberlein, K., Criswell, T., Pink, J. J., Kinsella, T. J. & Boothman, D. A. (2000) Proc. Natl. Acad. Sci. USA 97, 5907-5912.

21. Crawford, E. D., Eisenberger, M. A., McLeod, D. G., Spaulding, J. T., Benson, R., Dorr, F. A., Blumenstein, B. A., Davis, M. A. & Goodman, P. J. (1989) N. Engl. J. Med. 321, 419-424.

22. Bems, E. M., de Boer, W. & Mulder, E. (1986) Prostate 9, 247-259.

23. Ikonen, T., Palvimo, J. J. & Janne, O. A. (1997) J. Biol. Chem. 272, 29821-29828.

24. Yeh, S. & Chang, C. (1996) Proc. Natl. Acad. Sci. USA 93, 5517-5521.

25. Chang, C., Saltzman, A., Yeh, S., Young, W., Keller, E., Lee, H. J., Wang, C. & Mizokami, A. (1995) Crit. Rev. Eukaryotic Gene Expression 5, 97-125.

26. Ting, H. J., Yeh, S., Nishimura, K. & Chang, C. (2002) Proc. Natl. Acad. Sci. USA 99, 661-666.

27. Bouteille, M. (1971) Exp. Cell Res. 69, 135-147.

28. Fleshner, N. E. & Kucuk, 0. (2001) Urology 57, 90-94.

29. Menter, D. G., Sabichi, A. L. & Lippman, S. M. (2000) Cancer Epidemiol. Biomarkers Prev. 9, 1171-1182.

30. Veldscholte, J., Voorhorst-Ogink, M. M., Bolt-de Vries, J., van Rooij, H. C., Trapman, J. & Mulder, E. (1990) Biochim. Biophys. Acta 1052, 187-194.

31. Brinkmann, A. O., Kuiper, G. G., Ris-Stalpers, C., van Rooij, H. C., Romalo, G., Trifiro, M., Mulder, E., Pinsky, L., Schweikert, H. U. & Trapman, J. (1991) J. Steroid Biochem. Mol. Biol. 40, 349-352.

32. Horoszewicz, J. S., Kawinski, E. & Murphy, G. P. (1987) Anticancer Res. 7, 927-935.

33. Yeh, S., Miyamoto, H. & Chang, C. (1997) Lancet 349, 852-853.

34. Sadi, M. V., Walsh, P. C. & Barrack, E. R. (1991) Cancer 67, 3057-3064.

35. Stocker, A., Zimmer, S., Spycher, S. E. & Azzi, A. (1999) IUBMB Life 48, 49-55.

36. Zimmer, S., Stocker, A., Sarbolouki, M. N., Spycher, S. E., Sassoon, J. & Azzi, A. (2000) J. Biol. Chem. 275, 25672-25680.

37. Israel, K., Yu, W., Sanders, B. G. & Kline, K. (2000) Nutr. Cancer 36, 90-100.

38. Zhao, X. Y. & Feldman, D. (2001) Steroids 66, 293-300.

39. Beer, T. M., Hough, K. M., Garzotto, M., Lowe, B. A. & Henner, W. D. (2001) Semin. Oncol. 28, 49-55.

40. Clark, L. C., Combs, G. F., Jr., Turnbull, B. W., Slate, E. H., Chalker, D. K, Chow, J., Davis, L. S., Glover, R. A., Graham, G. F., Gross, E. G., et al. (1996) J. Am. Med. Assoc. 276, 1957-1963.

41. Clark, L. C., Cantor, K. P. & Allaway, W. H. (1991) Arch Environ. Health 46, 37-42.

42. Schrauzer, G. N. (2001) J. Am. Coll. Nutr. 20, 1-4.

43. Xing, N., Chen, Y., Mitchell, S. H. & Young, C. Y. (2001) Carcinogenesis 22, 409-414.

44. Zhu, W., Zhang, J. S. & Young, C. Y. (2001) Carcinogenesis 22, 1399-1403.

45. Chomczynski, P. (1993) BioTechniques 15, 532-537.

1. A pharmaceutical composition comprising vitamin E succinate or derivative and an anti-prostate cancer compound.

2. The pharmaceutical composition of claim 1, wherein the anti-prostate cancer compound is an antiandrogen.

3. The pharmaceutical composition of claim 2, wherein the anti-androgen is Flutamide, Casodex, or Nilutamide.

4. The pharmaceutical composition of claim 2, wherein the anti-androgen is Flutamide.

5. The pharmaceutical composition of claim 2, wherein the concentration of the anti-androgen is less than or equal to 20 uM.

6. The pharmaceutical composition of claim 1, wherein the vitamin E succinate has the structure shown in Formula 2.

7. The pharmaceutical composition of claim 1, wherein the concentration of the vitamin E succinate is less than or equal to 100 uM.

8. The pharmaceutical composition of claim 1, wherein the anti-prostate cancer compound is less than or equal to 20 uM.

9. A method of treating a subject with prostate cancer comprising administering the composition of claim 1.

10. The method of claim 9 wherein administering the composition comprises injecting the composition into the subject.

11. The method of claim 9, wherein administering the composition comprises taking the composition orally, taking by skin patch, or taking by subcutaneous injection.
